(19) United States
(12) Patent Application Publication
(10) Pub. No.: US 2018/0263712 A1
(43) Pub. Date: Sep. 20, 2018

(54) ENDOSCOPE POSITION SPECIFYING DEVICE, METHOD, AND PROGRAM

(71) Applicant: FUJIFILM Corporation, Tokyo (JP)

(72) Inventor: Yoshiro KITAMURA, Tokyo (JP)

(73) Assignee: FUJIFILM Corporation, Tokyo (JP)

(21) Appl. No.: 15/868,689

(22) Filed: Jan. 11, 2018

(30) Foreign Application Priority Data
Mar. 16, 2017 (JP) 2017-051505

(51) Int. Cl. A61B 34/20; A61B 5/06; A61B 1/267; A61B 1/04

(52) U.S. Cl. CPC A61B 34/20; A61B 5/065; A61B 2034/2057; A61B 1/041; A61B 1/2676

(57) ABSTRACT

A hole portion detection unit detects a hole portion of the bronchus from at least one of a first endoscope image or a second endoscope image temporally earlier than the first endoscope image. A first parameter calculation unit calculates a first parameter indicating the amount of parallel movement for matching the hole portions of the two endoscope images with each other. A second parameter calculation unit performs alignment between the two endoscope images based on the first parameter, and calculates a second parameter including the amount of enlargement and reduction. Based on the two parameters, a movement amount calculation unit calculates the amount of movement of the endoscope from the acquisition time of the second endoscope image to the acquisition time of the first endoscope image.

### CROSS REFERENCE TO RELATED APPLICATIONS

The present application claims priority under 35 U.S.C. § 119 to Japanese Patent Application No. 2017-051505 filed on Mar. 16, 2017. The above application is hereby expressly incorporated by reference, in its entirety, into the present application.

### BACKGROUND

### Field of the Invention

The present invention relates to an endoscope position specifying device, method, and program for specifying the position of an endoscope in a tubular structure having branch structures, such as a bronchus, in the case of observing the tubular structure by inserting the endoscope into the tubular structure.

### Description of the Related Art

In recent years, a technique of observing or treating a tubular structure, such as a bronchus and a large intestine of a patient, using an endoscope has been drawing attention. However, in the endoscope image, an image in which the color or texture of the inside of the tubular structure is clearly expressed by an imaging element, such as a charge coupled device (CCD), can be obtained, while the inside of the tubular structure is expressed as a two-dimensional image. For this reason, it is difficult to ascertain which position in the tubular structure the endoscope image represents. In particular, since a bronchial endoscope has a small diameter and accordingly has a narrow field of view, it is difficult to make the distal end of the endoscope reach a target position.

Therefore, a method has been proposed in which a virtual endoscope image similar to an image that has been actually captured by an endoscope is generated using a three-dimensional image acquired by tomographic imaging using a modality, such as a computed tomography (CT) apparatus or a magnetic resonance imaging (Mill) apparatus. The virtual endoscope image is used as a navigation image for guiding the endoscope to a target position in the tubular structure. However, even if the navigation image is used, in the case of a structure having a path branching in multiple stages, such as a bronchus, a skilled technique is required to make the distal end of the endoscope reach the target position in a short time. For this reason, a method has been proposed in which a bronchus image showing a graph structure of a bronchus, which is a tubular structure, is generated from a three-dimensional image and the position of an endoscope is indicated on the bronchus image while displaying the bronchus image (refer to JP2016-179121A).

In the case of indicating the position of the endoscope on the bronchus image as described above, it is necessary to accurately detect the amount of movement of the endoscope. Therefore, a method has been proposed in which an optical flow is calculated using a current endoscope image and a past endoscope image and the current position of the endoscope is estimated by using the optical flow (refer to JP2016-505279A). In addition, a method has been proposed in which the amount of movement of an endoscope is calculated based on the position of a characteristic structure characterizing a local part on the luminal mucosa included in the real endoscope image of preceding and subsequent imaging times, for example, the position of luminal mucosa wrinkles and blood vessels seen through the surface, using a capsule endoscope (refer to JP2014-000421A).

### SUMMARY

However, the optical flow has many parameters to be determined. Also in the method disclosed in JP2014-000421A, in order to calculate the amount of movement of the endoscope based on the characteristic structure, it is necessary to calculate the parameters, such as parallel movement and rotation. In position detection and movement amount calculation, as the number of parameters to be determined increases, a difference in matching increases, and the accuracy decreases. In order to increase the accuracy, the amount of calculation increases. This processing requires time.

The invention has been made in view of the above circumstances, and it is an object of the invention to more easily calculate the amount of movement of an endoscope inserted into a tubular structure having branch structures.

An endoscope position specifying device according to the invention comprises: endoscope image acquisition unit for sequentially acquiring endoscope images that are generated by an endoscope inserted into a tubular structure having a plurality of branch structures and that show an inner wall of the tubular structure; hole portion detection unit for detecting a hole portion of the tubular structure from at least one of a first endoscope image or a second endoscope image, which is acquired temporally before the first endoscope image, among the sequentially acquired endoscope images; first parameter calculation unit for calculating a first parameter indicating an amount of parallel movement of the first endoscope image with respect to the second endoscope image in order to match hole portions of the first and second endoscope images with each other; second parameter calculation unit for performing alignment between the first and second endoscope images based on the first parameter and calculating a second parameter including an amount of enlargement and reduction of the first endoscope image with respect to the second endoscope image in order to match the hole portions of the first and second endoscope images after the alignment with each other; and movement amount calculation unit for calculating an amount of movement of the endoscope from an acquisition time of the second endoscope image to an acquisition time of the first endoscope image based on the first and second parameters.

In the endoscope image acquired by the endoscope inserted into the tubular structure, the inner part of the tubular structure is dark since the light of the endoscope does not reach there. Accordingly, the inner part of the tubular structure has an appearance of a deep hole. The “hole portion of the tubular structure” means a region that is darker than the other regions in the endoscope image since the light of the endoscope does not reach there. The “amount of movement” includes the amount of movement calculated based on the first parameter and the amount of movement calculated based on the second parameter. The amount of movement calculated based on the first parameter is the amount of parallel movement, and the amount of movement calculated based on the second parameter is the amount of movement in a direction in which the tubular structure extends. As will be described later, in a case where the second parameter includes the amount of rotation, the amount of movement also includes the amount of rotational movement.

The endoscope position specifying device according to the invention may further comprise: image generation unit for generating an image of the tubular structure from a three-dimensional image including the tubular structure; and display control unit for displaying the image of the tubular structure and displaying a position of the endoscope based on the amount of movement on the image of the tubular structure.

In the endoscope position specifying device according to the invention, the display control unit may display the position of the endoscope by projecting the position of the endoscope in a direction in which the tubular structure in the image of the tubular structure extends.

The endoscope position specifying device according to the invention may further comprise: storage unit for storing the amount of movement; and deviation calculation unit for calculating a deviation of the endoscope within the tubular structure based on the stored amount of movement. The display control unit may display the position of the endoscope based on the deviation of the endoscope.

In the endoscope position specifying device according to the invention, the movement amount calculation unit may calculate the amount of movement by correcting at least one of the first parameter or the second parameter according to a diameter of the tubular structure.

In the endoscope position specifying device according to the invention, the second parameter calculation unit may calculate the second parameter further including an amount of rotation of the first endoscope image with respect to the second endoscope image.

An endoscope position specifying method according to the invention comprises: sequentially acquiring endoscope images that are generated by an endoscope inserted into a tubular structure having a plurality of branch structures and that show an inner wall of the tubular structure; detecting a hole portion of the tubular structure from at least one of a first endoscope image or a second endoscope image, which is acquired temporally before the first endoscope image, among the sequentially acquired endoscope images; calculating a first parameter indicating an amount of parallel movement of the first endoscope image with respect to the second endoscope image in order to match hole portions of the first and second endoscope images with each other; performing alignment between the first and second endoscope images based on the first parameter and calculating a second parameter including an amount of enlargement and reduction of the first endoscope image with respect to the second endoscope image in order to match the hole portions of the first and second endoscope images after the alignment with each other; and calculating an amount of movement of the endoscope from an acquisition time of the second endoscope image to an acquisition time of the first endoscope image based on the first and second parameters.

In addition, a program causing a computer to execute the endoscope position specifying method according to the invention may be provided.

Another endoscope position specifying device according to the invention comprises: a memory for storing a command to be executed by a computer; and a processor configured to execute the stored command. The processor executes: endoscope image acquisition processing for sequentially acquiring endoscope images that are generated by an endoscope inserted into a tubular structure having a plurality of branch structures and that show an inner wall of the tubular structure; hole portion detection processing for detecting a hole portion of the tubular structure from at least one of a first endoscope image or a second endoscope image, which is acquired temporally before the first endoscope image, among the sequentially acquired endoscope images; first parameter calculation processing for calculating a first parameter indicating an amount of parallel movement of the first endoscope image with respect to the second endoscope image in order to match hole portions of the first and second endoscope images with each other; second parameter calculation processing for performing alignment between the first and second endoscope images based on the first parameter and calculating a second parameter including an amount of enlargement and reduction of the first endoscope image with respect to the second endoscope image in order to match the hole portions of the first and second endoscope images after the alignment with each other; and movement amount calculation processing for calculating an amount of movement of the endoscope from an acquisition time of the second endoscope image to an acquisition time of the first endoscope image based on the first and second parameters.

According to the invention, endoscope images showing the inner wall of the tubular structure having branch structures are sequentially acquired, and the hole portion of the tubular structure is detected from at least one of the first endoscope image or the second endoscope image, which is acquired temporally earlier than the first endoscope image, among the sequentially acquired endoscope images. Then, the first parameter indicating the amount of parallel movement of the first endoscope image with respect to the second endoscope image is calculated to match the hole portions of the first and second endoscope images with each other. Alignment between the first and second endoscope images is performed based on the first parameter, and the second parameter including the amount of enlargement and reduction of the first endoscope image with respect to the second endoscope image is calculated to match the hole portions of the first and second endoscope images after the alignment with each other. Then, based on the first and second parameters, the amount of movement of the endoscope from the acquisition time of the second endoscope image to the acquisition time of the first endoscope image is calculated. As described above, according to the invention, the first parameter is calculated first, and the second parameter is calculated after aligning the first and second endoscope images based on the first parameter. Therefore, compared with a case where the first and second parameters are simultaneously calculated, it is possible to calculate the first and second parameters with a small amount of calculation. In addition, since the number of parameters to be processed is small, the calculated amount of movement does not largely deviate. As a result, it is possible to improve the reliability of the calculated amount of movement.

### BRIEF DESCRIPTION OF THE DRAWINGS

FIG. 1 is a hardware configuration diagram showing an outline of a diagnosis assistance system to which an endoscope position specifying device according to an embodiment of the invention is applied.

FIG. 2 is a diagram showing the schematic configuration of the endoscope position specifying device according to the present embodiment realized by installing an endoscope position specifying program on a computer.

FIG. 3 is a diagram showing an endoscope image.

FIG. 4 is a diagram illustrating the calculation of a deviation of an endoscope distal end.

FIG. 5 is a diagram illustrating the projection of the endoscope distal end onto a bronchus image.

FIG. 6 is a diagram showing an image displayed on a display.

FIG. 7 is a flowchart showing a process performed in the present embodiment.

FIG. 8 is a diagram illustrating the deviation of the endoscope within the bronchus.

### DESCRIPTION OF THE PREFERRED EMBODIMENTS

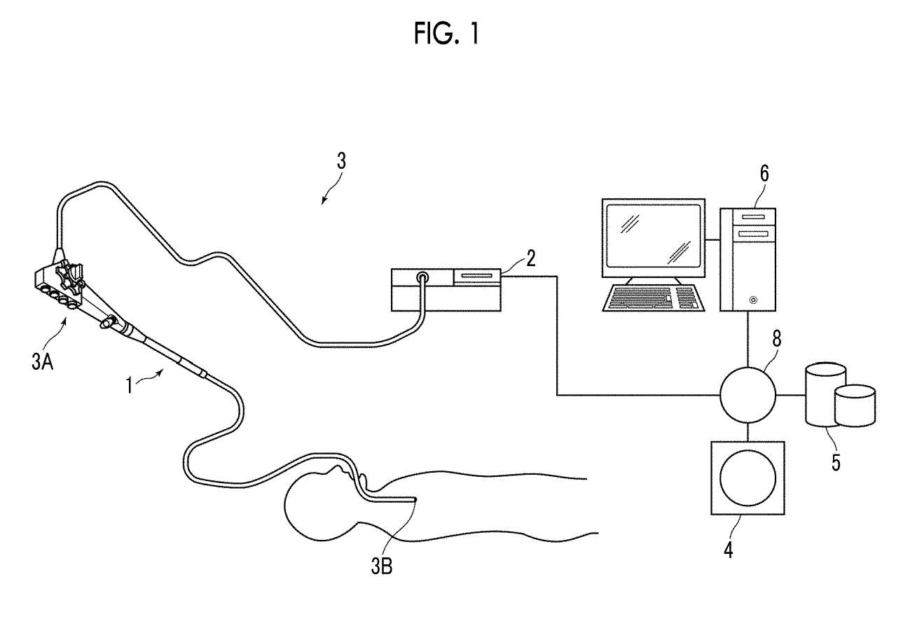

Hereinafter, embodiments of the invention will be described with reference to the accompanying diagrams. FIG. 1 is a hardware configuration diagram showing the outline of a diagnostic assistance system to which an endoscope position specifying device according to an embodiment of the invention is applied. As shown in FIG. 1, in this system, an endoscope apparatus 3, a three-dimensional image capturing apparatus 4, an image storage server 5, and an endoscope position specifying device 6 are connected to each other in a communicable state through a network 8.

The endoscope apparatus 3 includes an endoscope scope 1 for imaging the inside of a tubular structure of a subject, a processor device 2 for generating an image of the inside of the tubular structure based on a signal obtained by imaging, and the like.

The endoscope scope 1 is obtained by attaching an insertion part, which is inserted into the tubular structure of the subject, to an operation unit 3A, and is connected to the processor device 2 through a universal cord detachably connected to the processor device 2. The operation unit 3A includes various buttons for giving an instruction for an operation to make a distal end 3B of the insertion part curve in a vertical direction and a horizontal direction within a predetermined angular range, or for collecting samples of tissues by operating an insertion needle attached to the distal end of the endoscope scope 1, or for spraying a medicine. In the present embodiment, the endoscope scope 1 is a flexible mirror for bronchi, and is inserted into the bronchus of the subject. Then, light guided through an optical fiber from a light source device (not shown) provided in the processor device 2 is emitted from the distal end 3B of the insertion part of the endoscope scope 1, and an image of the inside of the bronchus of the subject is acquired by the imaging optical system of the endoscope scope 1. In order to facilitate the explanation, the distal end 3B of the insertion part of the endoscope scope 1 will be referred to as an endoscope distal end 3B in the following explanation.

The processor device 2 generates an endoscope image G0 by converting an imaging signal captured by the endoscope scope 1 into a digital image signal and correcting the image quality by digital signal processing, such as white balance adjustment and shading correction. The generated image is a moving image configured to include a plurality of endoscope images G0 expressed at a predetermined frame rate, such as 30 fps. The endoscope image G0 is transmitted to the image storage server 5 or the endoscope position specifying device 6.

The three-dimensional image capturing apparatus 4 is an apparatus that generates a three-dimensional image V0 showing a part, which is an examination target part of a subject, by imaging the part. Specifically, the three-dimensional image capturing apparatus 4 is a CT apparatus, an MRI apparatus, a positron emission tomography (PET) apparatus, an ultrasound diagnostic apparatus, or the like. The three-dimensional image V0 generated by the three-dimensional image capturing apparatus 4 is transmitted to the image storage server 5 and is stored therein. In the present embodiment, the three-dimensional image capturing apparatus 4 is a CT apparatus that generates the three-dimensional image V0 by imaging the chest including a bronchus.

The image storage server 5 is a computer that stores and manages various kinds of data, and includes a large-capacity external storage device and software for database management. The image storage server 5 transmits and receives image data and the like by performing communication with other apparatuses through the network 8. Specifically, the image storage server 5 acquires image data, such as the endoscope image G0 acquired by the endoscope apparatus 3 and the three-dimensional image V0 generated by the three-dimensional image capturing apparatus 4, through the network, and stores the image data in a recording medium, such as a large-capacity external storage device and manages the image data. The endoscope image G0 is moving image data sequentially acquired according to the movement of the endoscope distal end 3B. Therefore, it is preferable that the endoscope image G0 is transmitted to the endoscope position specifying device 6 without passing through the image storage server 5. The storage format of image data or the communication between apparatuses through the network 8 is based on protocols, such as a digital imaging and communication in medicine (DICOM).

The endoscope position specifying device 6 is realized by installing an endoscope position specifying program of the present embodiment on one computer. The computer may be a workstation or a personal computer that is directly operated by a doctor who performs diagnosis, or may be a server computer connected to these through a network. The endoscope position specifying program is distributed by being recorded on a recording medium, such as a digital versatile disc (DVD) or a compact disc read only memory (CD-ROM), and is installed onto the computer from the recording medium. Alternatively, the endoscope position specifying program is stored in a storage device of a server computer connected to the network or in a network storage so as to be accessible from the outside, and is downloaded and installed onto a computer used by a doctor, who is a user of the endoscope position specifying device 6, when necessary.

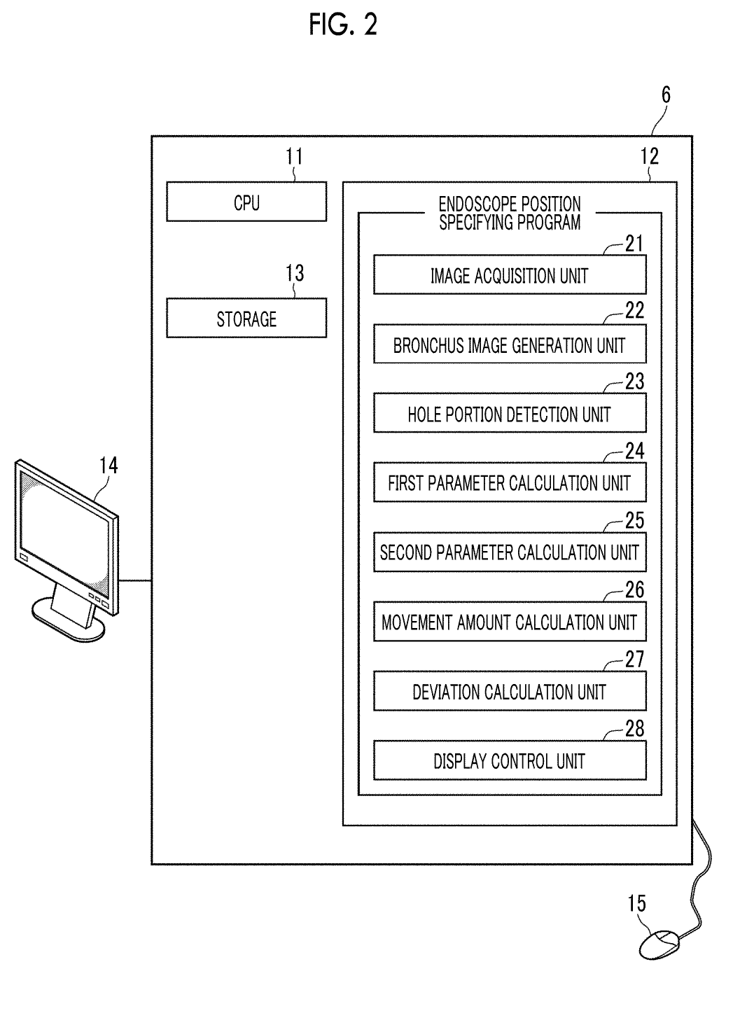

FIG. 2 is a diagram showing the schematic configuration of an endoscope position specifying device realized by installing an endoscope position specifying program on a computer. As shown in FIG. 2, the endoscope position specifying device 6 includes a central processing unit (CPU) 11, a memory 12, and a storage 13 as the configuration of a standard workstation. A display 14 and an input unit 15, such as a mouse, are connected to the endoscope position specifying device 6.

The endoscope image G0 and the three-dimensional image V0, which are acquired from the endoscope apparatus 3, the three-dimensional image capturing apparatus 4, the image storage server 5, and the like through the network 8, and the image generated by the processing in the endoscope position specifying device 6, and the like are stored in the storage 13.

The endoscope position specifying program is stored in the memory 12. As processing to be executed by the CPU 11, the endoscope position specifying program defines: image acquisition processing for sequentially acquiring the endoscope image G0 generated by the processor device 2 and acquiring image data, such as the three-dimensional image V0 generated by the three-dimensional image capturing apparatus 4; bronchus image generation processing for generating a bronchus image, which is an image of a tubular structure, from the three-dimensional image V0; hole portion detection processing for detecting a hole portion of the bronchus from at least one of a first endoscope image or a second endoscope image, which is acquired temporally earlier than the first endoscope image, among the sequentially acquired endoscope images; first parameter calculation processing for calculating a first parameter indicating the amount of parallel movement of the first endoscope image with respect to the second endoscope image in order to match the hole portions of the first and second endoscope images with each other; second parameter calculation processing for performing alignment between the first and second endoscope images based on the first parameter and calculating a second parameter including the amount of enlargement and reduction of the first endoscope image with respect to the second endoscope image in order to match the hole portions of the first and second endoscope images after the alignment with each other; movement amount calculation processing for calculating the amount of movement of the endoscope from the acquisition time of the second endoscope image to the acquisition time of the first endoscope image based on the first and second parameters; deviation calculation processing for calculating a deviation of the endoscope within the bronchus based on the amount of movement stored as will be described later; and display control processing for displaying the bronchus image and displaying the position of the endoscope on the bronchus image based on the amount of movement.

The CPU 11 executes these processes according to the program, so that the computer functions as an image acquisition unit 21, a bronchus image generation unit 22, a hole portion detection unit 23, a first parameter calculation unit 24, a second parameter calculation unit 25, a movement amount calculation unit 26, a deviation calculation unit 27, and a display control unit 28. The endoscope position specifying device 6 may include a plurality of processors that perform image acquisition processing, bronchus image generation processing, hole portion detection processing, first parameter calculation processing, second parameter calculation processing, movement amount calculation processing, deviation calculation processing, and display control processing. Here, the image acquisition unit 21 corresponds to endoscope image acquisition unit, and the bronchus image generation unit 22 corresponds to an image generation unit.

The image acquisition unit 21 sequentially acquires the endoscope image G0 by imaging the inside of the bronchus using the endoscope apparatus 3, and acquires the three-dimensional image V0. In a case where the three-dimensional image V0 is already stored in the storage 13, the image acquisition unit 21 may acquire the three-dimensional image V0 from the storage 13. The endoscope image G0 is displayed on the display 14. The image acquisition unit 21 stores the acquired endoscope image G0 and the acquired three-dimensional image V0 in the storage 13.

The bronchus image generation unit 22 generates a bronchus image from the three-dimensional image V0. Therefore, the bronchus image generation unit 22 generates a three-dimensional bronchus image by extracting a graph structure of a bronchial region included in the three-dimensional image V0 using the method disclosed in JP2010-220742A or the like, for example. Hereinafter, an example of the graph structure extraction method will be described.

In the three-dimensional image V0, pixels inside the bronchus are expressed as a region showing low pixel values since the pixels correspond to an air region. However, the bronchial wall is expressed as a cylindrical or linear structure showing relatively high pixel values. Therefore, the bronchus is extracted by performing structural analysis of the shape based on the distribution of pixel values for each pixel.

The bronchus branches in multiple stages, and the diameter of the bronchus decreases as the distance from the distal end decreases. The bronchus image generation unit 22 generates a plurality of three-dimensional images with different resolutions by performing multi-resolution conversion of the three-dimensional image V0 so that bronchi having different sizes can be detected, and applies a detection algorithm for each three-dimensional image of each resolution, thereby detecting tubular structures having different sizes.

First, at each resolution, a Hessian matrix of each pixel of the three-dimensional image is calculated, and it is determined whether or not the pixel is a pixel in the tubular structure from the magnitude relationship of eigenvalues of the Hessian matrix. The Hessian matrix is a matrix having, as its elements, partial differential coefficients of the second order of density values in directions of the respective axes (x, y, and z axes of the three-dimensional image), and is a 3×3 matrix as in the following Equation (1).

$${∇}^{2} I = [ \begin{matrix}{I}_{xx} & {I}_{xy} & {U}_{xz} \\ {I}_{xx} & {I}_{xy} & {I}_{xz} \\ {I}_{xx} & {I}_{xy} & {I}_{xz}\end{matrix} ] \\ {I}_{xx} = \frac{{δ}^{2} I}{δ {x}^{2}} , {I}_{xy} = \frac{{δ}^{2} I}{δ x δ {y}^{2}} , ⋯ \qquad ( 1 )$$

Assuming that the eigenvalues of the Hessian matrix at an arbitrary pixel are λ1, λ2, and λ3, it is known that the pixel is a tubular structure in a case where two of the eigenvalues are large and one eigenvalue is close to 0, for example, in a case where λ3, λ2>>λ1, and λ1≅0 are satisfied. In addition, an eigenvector corresponding to the minimum eigenvalue (λ1≅0) of the Hessian matrix matches a main axis direction of the tubular structure.

The bronchus can be expressed in a graph structure, but the tubular structure extracted in this manner is not necessarily detected as one graph structure, in which all tubular structures are connected to each other, due to the influence of a tumor or the like. Therefore, after the detection of the tubular structure from the three-dimensional image V0 is ended, by performing evaluation regarding whether each extracted tubular structure is within a predetermined distance and an angle between the direction of the basic line connecting arbitrary points on the two extracted tubular structures to each other and the main axis direction of each tubular structure is within a predetermined angle, it is determined whether or not a plurality of tubular structures are connected to each other, thereby reconstructing the connection relationship of the extracted tubular structures. By this reconstruction, the extraction of the graph structure of the bronchus is completed.

Then, the bronchus image generation unit 22 generates a three-dimensional graph structure showing the bronchi as a bronchus image by classifying the extracted graph structure into a start point, an end point, a branch point, and a side and connecting the start point, the end point, and the branch point to each other with the side. The bronchus image generation method is not limited to the method described above, and other methods may be adopted.

The bronchus image generation unit 22 detects the central axis of the graph structure of the bronchus. The distance from each pixel position on the central axis of the graph structure of the bronchus to the inner wall of the graph structure of the bronchus is calculated as the radius of the bronchus at the pixel position. The direction in which the central axis of the graph structure extends is a direction in which the bronchus extends.

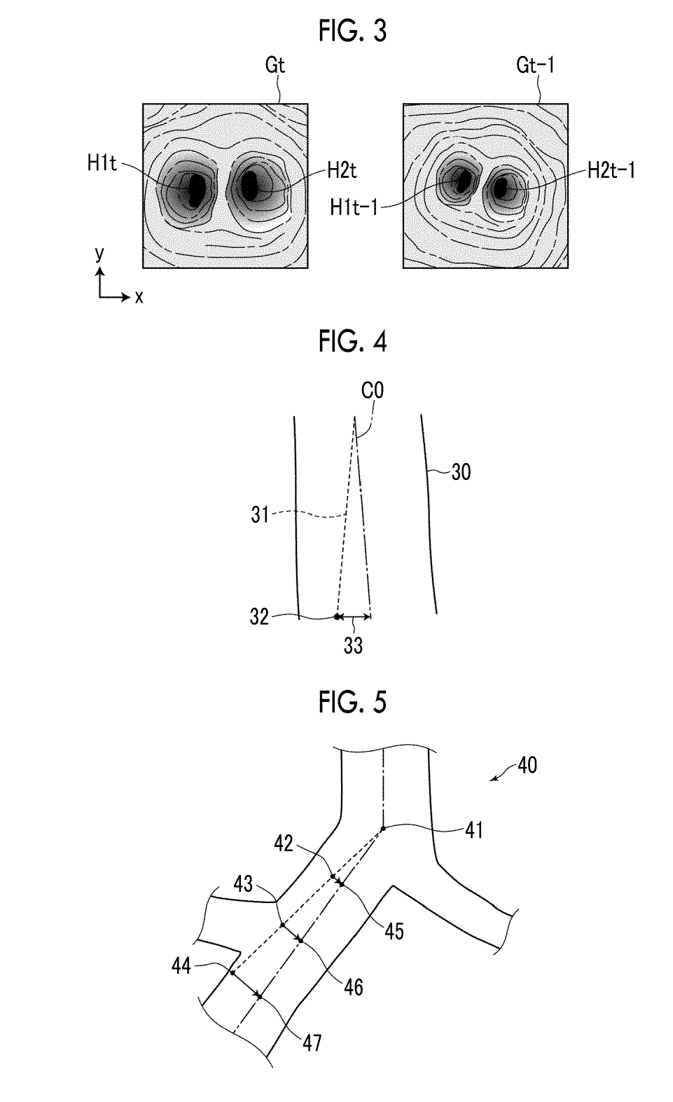

The hole portion detection unit 23 detects a hole portion of the bronchus from at least one of a first endoscope image or a second endoscope image, which is acquired temporally earlier than the first endoscope image, among the sequentially acquired endoscope images G0. In the present embodiment, explanation will be given on the assumption that the hole portion of the bronchus is detected from each of the first and second endoscope images. In the following explanation, reference numerals of the first and second endoscope images are Gt and Gt−1. Therefore, the second endoscope image Gt−1 is acquired at a time immediately before the first endoscope image Gt. FIG. 3 is a diagram showing first and second endoscope images. In a case where the first endoscope image Gt and the second endoscope image Gt−1 are compared with each other, the second endoscope image Gt−1 is acquired temporally earlier than the first endoscope image Gt. Therefore, two hole portions H1t−1 and H2t−1 at the branch of the bronchus included in the second endoscope image Gt−1 are smaller than two hole portions H1t and H2t included in the first endoscope image Gt.

The hole portion detection unit 23 detects hole portions from the first endoscope image Gt and the second endoscope image Gt−1 using the MSER method. In the MSER method, a dark region where the brightness is less than the threshold value in the endoscope image is detected. Then, a dark region where the brightness is less than the threshold value is detected while changing the threshold value. Then, in the MSER method, a threshold value at which the area of a dark region changes most largely with respect to a threshold value change is calculated, and a dark region where the brightness is less than the threshold value is detected as a hole portion.

The first parameter calculation unit 24 calculates a first parameter indicating the amount of parallel movement of the first endoscope image Gt with respect to the second endoscope image Gt−1 in order to match the hole portions of the first endoscope image Gt and the second endoscope image Gt−1 with each other. Specifically, the first parameter calculation unit 24 calculates a correlation while moving the first endoscope image Gt in a two-dimensional manner with respect to the second endoscope image Gt−1, with a state in which the center of gravity of the first endoscope image Gt and the center of gravity of the second endoscope image Gt−1 match each other being an initial position. Then, the two-dimensional amount of movement of the first endoscope image Gt having the maximum correlation is calculated as a first parameter P1. The first parameter P1 is x and y values in a case where the x axis is set in the horizontal direction and the y axis is set in the vertical direction on the paper surface as shown in FIG. 3.

The first parameter calculation unit 24 may extract a local region including a hole portion from each of the first endoscope image Gt and the second endoscope image Gt−1, and calculate the first parameter P1 only using the extracted region. Therefore, it is possible to reduce the amount of calculation for calculating the first parameter P1. In addition, in each of the first endoscope image Gt and the second endoscope image Gt−1, the first parameter P1 may be calculated by increasing the weighting of a local region including a hole portion.

The second parameter calculation unit 25 performs alignment between the first endoscope image Gt and the second endoscope image Gt−1 based on the first parameter P1, and calculates a second parameter P2 including the amount of enlargement and reduction of the first endoscope image Gt with respect to the second endoscope image Gt−1 in order to match the hole portions of the first endoscope image Gt and the second endoscope image Gt−1 after the alignment with each other. In the present embodiment, in addition to the amount of enlargement and reduction, the second parameter P2 further including the amount of rotation of the first endoscope image Gt with respect to the second endoscope image Gt−1 is calculated.

Therefore, the second parameter calculation unit 25 performs alignment between the first endoscope image Gt and the second endoscope image Gt−1 based on the first parameter P1 first. Specifically, the alignment is performed by moving the first endoscope image Gt in parallel to the second endoscope image Gt−1 based on the first parameter P1.

Then, the second parameter calculation unit 25 calculates a correlation while gradually enlarging and reducing the first endoscope image Gt after the alignment with respect to the second endoscope image Gt−1. In this case, in a case where the size of the hole portion included in the first endoscope image Gt matches the size of the hole portion included in the second endoscope image Gt−1, the correlation is maximized. The second parameter calculation unit 25 calculates the enlargement ratio of the first endoscope image Gt having the maximum correlation as the amount of enlargement and reduction included in the second parameter P2.

The second parameter calculation unit 25 calculates a correlation while gradually rotating the first endoscope image Gt after the alignment with respect to the second endoscope image Gt−1 with the center of the detected hole portion as a reference. In this case, in a case where there are a plurality of detected hole portions, the second parameter calculation unit 25 calculates a correlation while gradually rotating the first endoscope image Gt after the alignment with respect to the second endoscope image Gt−1 with the center of each of the detected hole portions as a reference. The correlation may also be calculated with only the center of one detected hole portion as a reference. Then, the rotation angle of the first endoscope image Gt at the time at which the correlation is maximized is calculated as the amount of rotation included in the second parameter P2. The second parameter calculation unit 25 may first calculate any of the amount of enlargement and reduction and the amount of rotation included in the second parameter P2.

Based on the first parameter P1 and the second parameter P2, the movement amount calculation unit 26 calculates the amount of movement of the endoscope distal end 3B from the acquisition position of the second endoscope image Gt−1 to the acquisition position of the first endoscope image Gt. Specifically, the amount of parallel movement of the endoscope distal end 3B, the amount of movement of the endoscope distal end 3B in a direction in which the central axis of the bronchus extends, and the amount of rotational movement of the endoscope distal end 3B are calculated. Therefore, the movement amount calculation unit 26 first sets the initial position of the endoscope distal end 3B in the bronchus image extracted by the bronchus image generation unit 22. In the present embodiment, the initial position is the position of the first branch in the endoscope image G0 displayed on the display 14. For the setting of the initial position, the display control unit 28 displays the bronchus image extracted by the bronchus image generation unit 22 extracted on the display 14. The operator sets the initial position on the bronchus image displayed on the display 14 using the input unit 15. The initial position may be automatically set on the bronchus image by matching the endoscope image G0 at the position of the first branch with the bronchus image.

In the present embodiment, with the initial position as a start position, the amount of movement is calculated every time the endoscope image G0 is acquired. Here, the calculation of the amount of movement using the first endoscope image Gt and the second endoscope image Gt−1 at a certain point in time will be described. The movement amount calculation unit 26 calculates the amount of movement by converting the first parameter P1 and the second parameter P2 into the amount of movement of the endoscope distal end 3B. Here, the acquisition position of the second endoscope image Gt−1 is specified by the immediately preceding process in which the second endoscope image Gt−1 is the first endoscope image Gt. The movement amount calculation unit 26 acquires the radius of the bronchus at the acquisition position of the second endoscope image Gt−1 from the bronchus image. Then, the movement amount calculation unit 26 calculates the amount of parallel movement of the endoscope distal end 3B by multiplying the first parameter P1, which is the amount of parallel movement, by the acquired radius of the bronchus as a scaling coefficient. In addition, by multiplying the amount of enlargement and reduction included in the second parameter P2 by the scaling coefficient, the amount of movement of the endoscope distal end 3B in a direction in which the central axis of the bronchus extends is calculated. In a case where the amount of enlargement and reduction is an enlargement value (that is, in a case where the enlargement ratio is larger than 1), the direction of movement along the central axis of the bronchus is a direction in which the endoscope distal end 3B faces. In a case where the amount of enlargement and reduction is a reduction value (that is, in a case where the enlargement ratio is smaller than 1), the direction of movement along the central axis of the bronchus is a direction opposite to the direction in which the endoscope distal end 3B faces. For the amount of rotation included in the second parameter P2, the amount of rotation is calculated as the amount of rotational movement as it is without being multiplied by the scaling coefficient.

The endoscope distal end 3B moves freely within the bronchus in actual examination. However, in a case where the degree of freedom of movement is high, it is difficult to specify the position of the endoscope distal end 3B. Here, in the examination using an endoscope, it is important to notify the operator of the part of the bronchus at which the endoscope distal end 3B is located. In the present embodiment, a condition that the endoscope distal end 3B moves along the central axis C0 of the bronchus is added, and the position of the endoscope distal end 3B is specified under the condition. Therefore, the movement amount calculation unit 26 stores the amount of movement, that is, the amount of parallel movement of the endoscope distal end 3B, the amount of movement of the endoscope distal end 3B in a direction in which the central axis of the bronchus extends, and the amount of rotational movement of the endoscope distal end 3B, in the storage 13. In the present embodiment, the amount of movement is accumulated and stored every time the endoscope image G0 is acquired from the initial position.

The deviation calculation unit 27 calculates the deviation of the endoscope distal end 3B within the bronchus based on the amount of movement stored in the storage 13. FIG. 4 is a diagram illustrating the calculation of the deviation of the endoscope distal end 3B. A bronchus 30 and its central axis C0 are shown in FIG. 4. In practice, the endoscope distal end 3B moves with a distance from the central axis C0 as indicated by a broken line 31. In the present embodiment, based on the amount of parallel movement among the amounts of movement stored in the storage 13, the distance of the endoscope distal end 3B from the central axis C0 is calculated as the deviation of the endoscope distal end 3B within the bronchus. As shown in FIG. 4, in a case where the endoscope distal end 3B is located at a position 32, the deviation is expressed by 33.

The display control unit 28 projects the position of the endoscope distal end 3B onto the central axis in the bronchus image based on the amount of movement of the endoscope distal end 3B from the acquisition position of the second endoscope image Gt−1 to the acquisition position of the first endoscope image Gt and the deviation of the endoscope distal end 3B calculated by the deviation calculation unit 27. FIG. 5 is a diagram illustrating the projection of the endoscope distal end 3B onto the bronchus image. In FIG. 5, the initial position of the endoscope distal end 3B in a bronchus image 40 is set as a position 41. As the endoscope distal end 3B moves from the initial position 41 toward the back of the bronchus with a deviation with respect to a position 42, a position 43, and a position 44, the position 42, the position 43, and the position 44 are projected to a position 45, a position 46, and a position 47, respectively. The display control unit 28 connects the position of the endoscope distal end 3B projected onto the central axis C0 and displays the result on the bronchus image 40 displayed on the display 14.

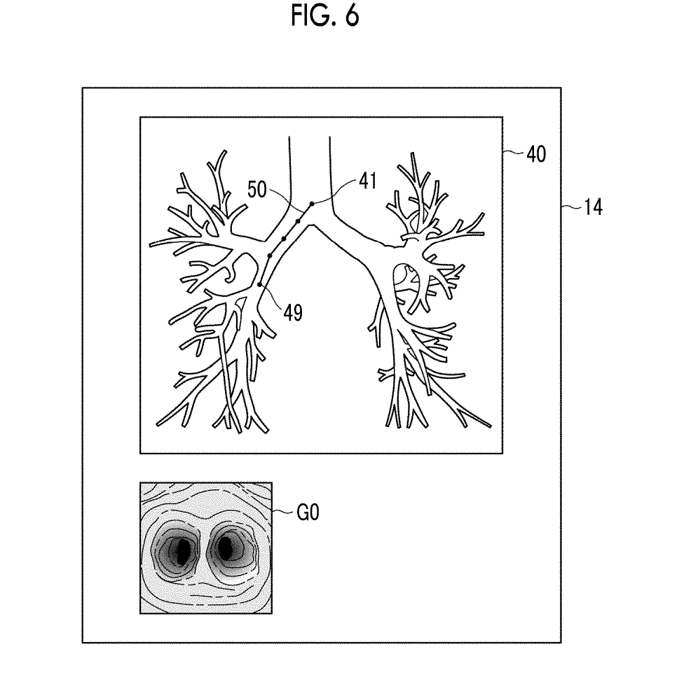

FIG. 6 is a diagram showing a bronchus image displayed on the display. As shown in FIG. 6, the bronchus image 40 and the endoscope image G0 captured at the current position are displayed on the display 14. The endoscope image G0 is the first endoscope image Gt. In the bronchus image 40, the initial position 41, a position 49 of the endoscope distal end 3B, and a trajectory 50 up to the current position obtained by connecting the projection position of the endoscope distal end 3B between the initial position 41 and the position 49 are displayed. The distal end of the trajectory 50 is the current position of the endoscope distal end 3B. In addition, for example, the current position of the endoscope distal end 3B may blink or a mark may be given thereto, so that the position of the endoscope distal end 3B can be viewed in the bronchus image 40.

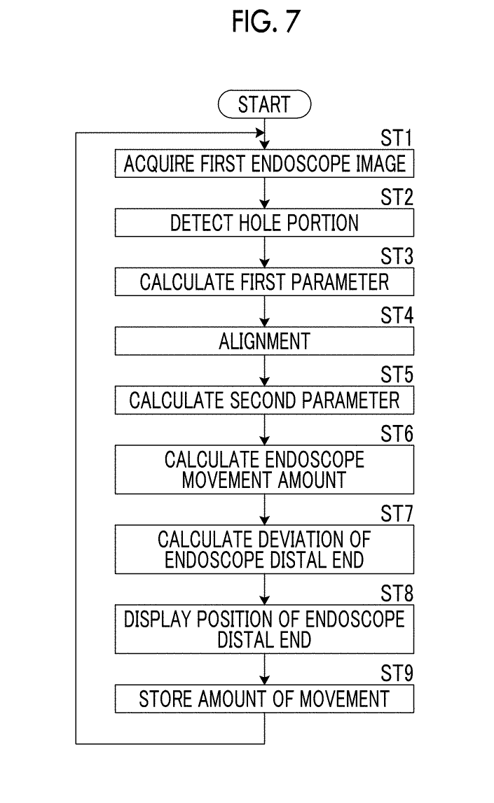

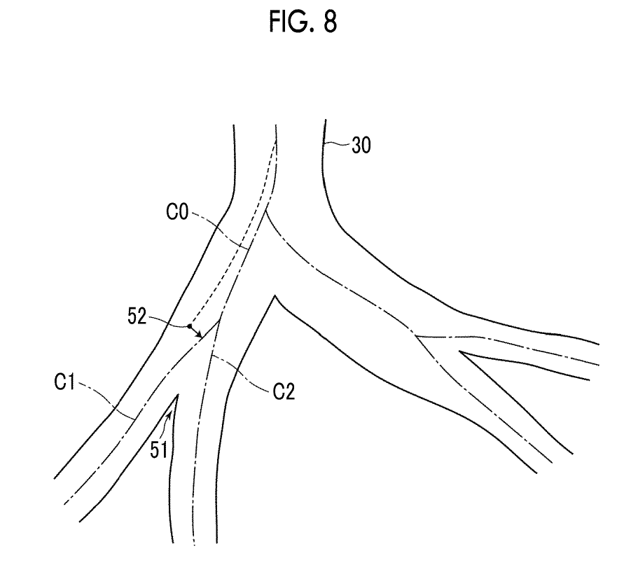

Next, a process performed in the present embodiment will be described. FIG. 7 is a flowchart showing the process performed in the present embodiment. Here, the process in a case where the endoscope distal end 3B is inserted from the initial position 41 toward the back of the bronchus and the endoscope image G0 at a certain point in time is the first endoscope image Gt will be described. In addition, it is assumed that a bronchus image is generated from the three-dimensional image V0 by the bronchus image generation unit 22. The image acquisition unit 21 acquires the endoscope image G0 at a certain point in time as the first endoscope image Gt (step ST1), and the hole portion detection unit 23 detects a hole portion of the bronchus from each of the first endoscope image Gt and the second endoscope image Gt−1 acquired temporally earlier than the first endoscope image Gt (step ST2).

Then, the first parameter calculation unit 24 calculates a first parameter indicating the amount of parallel movement of the first endoscope image Gt with respect to the second endoscope image Gt−1 in order to match the hole portions of the first endoscope image Gt and the second endoscope image Gt−1 with each other (step ST3). Then, the second parameter calculation unit 25 performs alignment between the first endoscope image Gt and the second endoscope image Gt−1 based on the first parameter P1 (step ST4), and calculates a second parameter including the amount of enlargement and reduction of the first endoscope image Gt with respect to the second endoscope image Gt−1 in order to match the hole portions of the first endoscope image Gt and the second endoscope image Gt−1 after the alignment with each other (step ST5).

Then, based on the first parameter P1 and the second parameter P2, the movement amount calculation unit 26 calculates the amount of movement of the endoscope from the acquisition time of the second endoscope image Gt−1 to the acquisition time of the first endoscope image Gt (step ST6). Then, the deviation calculation unit 27 calculates a deviation of the endoscope distal end 3B (step ST7). Then, based on the amount of movement and the deviation of the endoscope distal end 3B, the display control unit 28 displays the position of the endoscope distal end 3B on the bronchus image 40 displayed on the display 14 (step ST8). Then, the movement amount calculation unit 26 stores the amount of movement in the storage 13 (step ST9), and the process returns to step ST1.

As described above, in the present embodiment, the first parameter P1 is calculated first, and the second parameter P2 is calculated after aligning the first and second endoscope images Gt and Gt−1 based on the first parameter P1. Therefore, compared with a case where the first and second parameters P1 and P2 are simultaneously calculated, it is possible to calculate the first and second parameters P1 and P2 with a small amount of calculation. In addition, since the number of parameters to be processed is small, the calculated amount of movement does not largely deviate. As a result, it is possible to improve the reliability of the calculated amount of movement.

By displaying the bronchus image 40 and displaying information indicating the position of the endoscope on the bronchus image 40, it is possible to easily check the position of the endoscope within the bronchus.

In addition, by calculating the amount of movement by correcting the first and second parameters P1 and P2 according to the diameter of the bronchus, it is possible to calculate the amount of movement reflecting the amount of movement of the actual endoscope.

In the present embodiment, since the endoscope distal end 3B is projected onto the central axis of the bronchus image, the position of the endoscope distal end 3B on the central axis of the bronchus is displayed in the displayed bronchus image. However, at the branch of the bronchus, the central axis is divided into two central axes. FIG. 7 is a diagram illustrating the deviation of the endoscope within the bronchus. As shown in FIG. 7, the central axis C0 of the bronchus 30 is divided into two central axes C1 and C2 at a branch 51 thereafter. In the present embodiment, the deviation calculation unit 27 calculates the deviation of the endoscope distal end 3B within the bronchus. By calculating the deviation in this manner, it is possible to calculate the position of the actual endoscope distal end 3B within the bronchus. Therefore, even in a case where the central axis C0 is divided into the central axis C1 and the central axis C2 by the branch 51, it is possible to know the position 52 of the endoscope distal end 3B based on the deviation. As a result, in a case where the endoscope distal end 3B is located at the position 52, a central axis whose position 52 is to be projected can be determined as the central axis C1. Therefore, it is possible to accurately specify the position of the endoscope within the bronchus.

In the embodiment described above, the amount of movement is accumulated and stored in the storage 13 every time the endoscope image G0 is acquired from the initial position 41. Here, the amount of movement is accumulated and stored in order to determine in which direction the endoscope distal end 3B is directed at the branch of the bronchus. Therefore, the accumulated amount of movement may be reset to 0 every time the endoscope distal end 3B passes the branch, and the amount of movement may be accumulated and stored only from the passed branch to the next branch.

In the embodiment described above, the hole portion detection unit 23 detects a hole portion from each of the first and second endoscope images. However, a hole portion may also be detected from one of the first and second endoscope images Gt and Gt−1. For example, in a case where a hole portion is detected only from the first endoscope image Gt, an image in which the detected hole portion is cut out or an image in which the weight of the hole portion is increased can be generated, and the first parameter P1 and the second parameter P2 can be calculated by using such an image and the second endoscope image Gt−1.

In the embodiment described above, the second parameter P2 includes the amount of rotation. However, the second parameter P2 including only the amount of enlargement and reduction may be calculated.

In the embodiment described above, the deviation of the endoscope is calculated based on the stored amount of movement, and the position of the endoscope is displayed based on the amount of movement and the deviation. However, the position of the endoscope may be displayed based on only the amount of movement without calculating the deviation of the endoscope.

In the above embodiment, the case has been described in which the endoscope position specifying device of the invention is applied to the observation of the bronchus. However, without being limited thereto, the invention can also be applied to a case of observing a tubular structure having branch structures, such as blood vessels, with an endoscope.

Hereinafter, the effect of the embodiment of the invention will be described.

By generating an image of a tubular structure from a three-dimensional image including a tubular structure of a subject, displaying the image of the tubular structure, and displaying information indicating the position of an endoscope on the image, it is possible to easily check the position of the endoscope within the tubular structure.

By calculating the amount of movement by correcting at least one of the first parameter or the second parameter according to the diameter of the extracted tubular structure, it is possible to calculate the amount of movement reflecting the amount of movement of the actual endoscope.

By making the amount of rotation of the first endoscope image with respect to the second endoscope image be included in the second parameter, it is also possible to calculate the amount of movement for the rotation of the endoscope.

By storing the amount of movement and calculating the deviation of the endoscope within the tubular structure based on the stored amount of movement, in a case where the endoscope passes the branch of the tubular structure, it can be estimated in which direction of the endoscope moves by referring to the deviation of the endoscope. Therefore, it is possible to accurately specify the position of the endoscope within the tubular structure.

What is claimed is:

1. An endoscope position specifying device, comprising:
endoscope image acquisition unit for sequentially acquiring endoscope images that are generated by an endoscope inserted into a tubular structure having a plurality of branch structures and that show an inner wall of the tubular structure;
hole portion detection unit for detecting a hole portion of the tubular structure from at least one of a first endoscope image or a second endoscope image, which is acquired temporally before the first endoscope image, among the sequentially acquired endoscope images;
first parameter calculation unit for calculating a first parameter indicating an amount of parallel movement of the first endoscope image with respect to the second endoscope image in order to match hole portions of the first and second endoscope images with each other;
second parameter calculation unit for performing alignment between the first and second endoscope images based on the first parameter and calculating a second parameter including an amount of enlargement and reduction of the first endoscope image with respect to the second endoscope image in order to match the hole portions of the first and second endoscope images after the alignment with each other; and
movement amount calculation unit for calculating an amount of movement of the endoscope from an acquisition time of the second endoscope image to an acquisition time of the first endoscope image based on the first and second parameters.

2. The endoscope position specifying device according to claim 1, further comprising:
image generation unit for generating an image of the tubular structure from a three-dimensional image including the tubular structure; and
display control unit for displaying the image of the tubular structure and displaying a position of the endoscope based on the amount of movement on the image of the tubular structure.

3. The endoscope position specifying device according to claim 2,
wherein the display control unit displays the position of the endoscope by projecting the position of the endoscope in a direction in which the tubular structure in the image of the tubular structure extends.

4. The endoscope position specifying device according to claim 3, further comprising:
storage unit for storing the amount of movement; and
deviation calculation unit for calculating a deviation of the endoscope within the tubular structure based on the stored amount of movement,
wherein the display control unit displays the position of the endoscope based on the deviation of the endoscope.

5. The endoscope position specifying device according to claim 1,
wherein the movement amount calculation unit calculates the amount of movement by correcting at least one of the first parameter or the second parameter according to a diameter of the tubular structure.

6. The endoscope position specifying device according to claim 1,
wherein the second parameter calculation unit calculates the second parameter further including an amount of rotation of the first endoscope image with respect to the second endoscope image.

7. An endoscope position specifying method, comprising:
sequentially acquiring endoscope images that are generated by an endoscope inserted into a tubular structure having a plurality of branch structures and that show an inner wall of the tubular structure;
detecting a hole portion of the tubular structure from at least one of a first endoscope image or a second endoscope image, which is acquired temporally before the first endoscope image, among the sequentially acquired endoscope images;
calculating a first parameter indicating an amount of parallel movement of the first endoscope image with respect to the second endoscope image in order to match hole portions of the first and second endoscope images with each other;
performing alignment between the first and second endoscope images based on the first parameter and calculating a second parameter including an amount of enlargement and reduction of the first endoscope image with respect to the second endoscope image in order to match the hole portions of the first and second endoscope images after the alignment with each other; and
calculating an amount of movement of the endoscope from an acquisition time of the second endoscope image to an acquisition time of the first endoscope image based on the first and second parameters.

8. A non-transitory computer-readable recording medium having stored therein an endoscope position specifying program causing a computer to execute:
a step of sequentially acquiring endoscope images that are generated by an endoscope inserted into a tubular structure having a plurality of branch structures and that show an inner wall of the tubular structure;
a step of detecting a hole portion of the tubular structure from at least one of a first endoscope image or a second endoscope image, which is acquired temporally before the first endoscope image, among the sequentially acquired endoscope images;
a step of calculating a first parameter indicating an amount of parallel movement of the first endoscope image with respect to the second endoscope image in order to match hole portions of the first and second endoscope images with each other;
a step of performing alignment between the first and second endoscope images based on the first parameter and calculating a second parameter including an amount of enlargement and reduction of the first endoscope image with respect to the second endoscope image in order to match the hole portions of the first and second endoscope images after the alignment with each other; and
a step of calculating an amount of movement of the endoscope from an acquisition time of the second endoscope image to an acquisition time of the first endoscope image based on the first and second parameters.
